(19) United States
(12) Patent Application Publication
(10) Pub. No.: US 2026/0091419 A1
(43) Pub. Date: Apr. 2, 2026

(54) ORGANIC WASTE TREATMENT METHOD AND ORGANIC WASTE TREATMENT SYSTEM

(71) Applicant: TOYOTA JIDOSHA KABUSHIKI KAISHA, Toyota-shi (JP)

(72) Inventor: Takema FURUKAWA, Toyota-shi (JP)

(73) Assignee: TOYOTA JIDOSHA KABUSHIKI KAISHA, Toyota-shi (JP)

(21) Appl. No.: 19/295,815

(22) Filed: Aug. 11, 2025

(30) Foreign Application Priority Data
Oct. 1, 2024 (JP) 2024-172165

(51) Int. Cl. B09B 3/60; B09B 101/70; C12P 5/02

(52) U.S. Cl. CPC B09B 3/60; C12P 5/023; B09B 2101/70

(57) ABSTRACT

An organic waste treatment method according to the present disclosure is an organic waste treatment method of performing methane fermentation of a mixture of poultry manure and food waste, in which the mixture contains the food waste at a ratio of 5% or more. With the organic waste treatment method, it is possible to stably perform the methane fermentation of the organic waste at low cost.

### CROSS-REFERENCE TO RELATED APPLICATION

This application claims priority to Japanese Patent Application No. 2024-172165 filed on Oct. 1, 2024. The disclosure of the above-identified application, including the specification, drawings, and claims, is incorporated by reference herein in its entirety.

### BACKGROUND

### 1. Technical Field

The present disclosure relates to an organic waste treatment method and an organic waste treatment system.

### 2. Description of Related Art

A method of treating organic waste by methane fermentation is known.

For example, Japanese Patent No. 4512823 (JP 4512823 B) discloses a technology for performing the methane fermentation of poultry manure.

### SUMMARY

The inventors have found the following problem about an organic waste treatment method and an organic waste treatment system.

Poultry manure contains uric acid, and therefore, has a high nitrogen concentration, so that uric acid sometime changes into ammonia in a fermentation vessel. The methane fermentation can be inhibited by ammonia. Therefore, for stably promoting the methane fermentation, a countermeasure such as the installation of an ammonia removal device that dilutes waste with water to decrease the nitrogen concentration is taken. However, in the case where waste is diluted with water, the amount of treated water increases, and therefore, the cost for the treatment of the treated water increases. Further, the size of the fermentation vessel increases, and therefore, equipment cost increases. Further, in the case where the ammonia removal device is installed, the cost for installing removal devices increases. Therefore, the development of a technology for stably performing the methane fermentation of the organic waste at low cost has been demanded.

The present disclosure has been made in view of this problem, and has an object to provide an organic waste treatment method and an organic waste treatment system that make it possible to stably perform the methane fermentation of the organic waste at low cost.

An aspect for achieving the above object is an organic waste treatment method of performing methane fermentation of a mixture of poultry manure and food waste, in which the mixture contains the food waste at a ratio of 5% or more.

An aspect for achieving the above object is an organic waste treatment method of performing methane fermentation of a mixture of poultry manure and chicken eggs, in which the mixture contains the chicken eggs at a ratio of less than 20%.

An aspect for achieving the above object is an organic waste treatment system that performs methane fermentation of a mixture of poultry manure and food waste, in which the mixture contains the food waste at a ratio of 5% or more.

An aspect for achieving the above object is an organic waste treatment system that performs methane fermentation of a mixture of poultry manure and chicken eggs, in which the mixture contains the chicken eggs at a ratio of less than 20%.

With the present disclosure, it is possible to provide an organic waste treatment method and an organic waste treatment system that make it possible to stably perform the methane fermentation of the organic waste at low cost.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a block diagram showing an exemplary configuration of an organic waste treatment system according to an embodiment;

FIG. 2 is a flowchart for describing an example of the flow of an organic waste treatment method according to the embodiment;

FIG. 3 is a graph showing methane generation amounts in the case of the methane fermentation of a mixture containing food waste; and

FIG. 4 is a graph showing methane generation amounts in the case of the methane fermentation of a mixture containing chicken eggs.

### DETAILED DESCRIPTION OF EMBODIMENTS

An embodiment of the present disclosure will be described below in detail with reference to the drawings. In the drawings, identical or corresponding elements are denoted by identical reference characters, and for clear descriptions, repetitive descriptions are omitted as necessary. Further, to facilitate understanding, scales of parts in the drawings are occasionally different from actual scales.

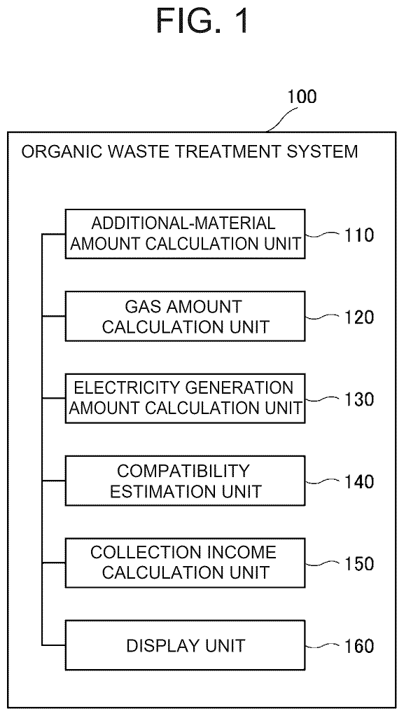

FIG. 1 is a block diagram showing an exemplary configuration of an organic waste treatment system 100 according to an embodiment. The organic waste treatment system 100 is a system that treats a mixture containing poultry manure and an additional material by methane fermentation. The additional material is food waste or chicken eggs. The food waste includes leftovers, cooking garbage, and the like, and is garbage that is discarded from restaurants, for example. The organic waste treatment system 100 is a system that calculates and presents the amount and kind of the additional material that needs to be added, depending on the amount of the poultry manure to be treated.

As shown in FIG. 1, the organic waste treatment system 100 includes at least an additional-material amount calculation unit 110 and a display unit 160, and furthermore, may include a gas amount calculation unit 120, an electricity generation amount calculation unit 130, a compatibility estimation unit 140, and a collection income calculation unit 150. The additional-material amount calculation unit 110 accepts the input of the amount of high-nitrogen-concentration organic waste to be treated, that is, the input of the amount of the poultry manure, and calculates the amount of the additional material to be added, based on Expression (1) described below. The additional-material amount calculation unit 110 calculates an additional-material amount for each kind of the additional material. In the case where the additional material is the food waste, the additional-material amount calculation unit 110 calculates the amount of the food waste to be added, such that the food waste is contained at a weight ratio of 5% or more in the mixture of the poultry manure and the food waste. In the case where the additional material is the chicken eggs, the additional-material amount calculation unit 110 calculates the amount of the chicken eggs to be added, such that the chicken eggs are contained at a weight ratio of less than 20% in the mixture of the poultry manure and the chicken eggs. The mixture in which the food waste is contained at a ratio of 5% or more and the mixture in which the chicken eggs are contained at a ratio of less than 20% allow the methane fermentation to be efficiently performed, and are preferable.

$$\begin{matrix}Expression 1 & \\ ( {α}_{i} + {β}_{a d d} ) \times TC / {TN}_{max} = \{ {( TC / TN )}_{i} \times {α}_{i} \} + \{ ( TC / {TN}_{) a d d} \times {β}_{a d d} \} & ( 1 )\end{matrix}$$

Here, TC represents a total carbo amount, and TN represents a total nitrogen amount. TC/TNₘₐₓ is a TC/TN ratio when a co-digestion performance index (CPI) is maximal. TC/TNᵢ is a TC/TN ratio of the high-nitrogen-concentration organic waste that is a substrate, that is, a TC/TN ratio of the poultry manure. TC/TN_{add} is a TC/TN ratio of the additional material to be added. Further, αᵢ is a predicted amount of the high-nitrogen-concentration organic waste. Further, β_{add} is the amount of the additional material to be added.

The gas amount calculation unit 120 calculates the amount of methane gas that is generated in the case where the methane fermentation of the mixture is performed, based on the additional-material amount calculated by the additional-material amount calculation unit 110 and Expression (2) described below. Furthermore, the gas amount calculation unit 120 may calculate the amount of methane gas that is generated in the case where the methane fermentation is performed without adding the additional material in the high-nitrogen-concentration organic waste. In this case, the gas amount calculation unit 120 may calculate an amount by which the methane gas is increased when the methane fermentation is performed with the addition of the additional material.

$$\begin{matrix}Expression 2 & \\ Total Methane ( {m}^{3} / g - VS ) = {CPI}_{TC / TN} \cdot \{ \{ {α}_{i} / ( {α}_{i} + {β}_{a d d} ) \} \cdot {α}_{i} \cdot {VS}_{ai} ( \% ) \cdot {α}_{gas} \} + \{ {β}_{a d d} / ( α i + {β}_{a d d} ) \cdot {β}_{a d d} \cdot {VS}_{β a d d} ( \% ) \cdot {β}_{g a s} \} & ( 2 )\end{matrix}$$

VS is a volatile substance. CPI_{TC/TN} is a co-digestion performance index that is predicted from the TC/TN ratio. VSₐᵢ is a VS proportion (%) in the high-nitrogen-concentration organic waste. Further, α_{gas} is a generated methane gas amount (m³/g-VS) in the case where the high-nitrogen-concentration organic waste is fermented alone. VS_{βadd} is a VS proportion (%) in the additional material to be added. Further, β_{gas} is a generated methane gas amount (m³/g-VS) in the case where the additional material to be added is fermented alone.

The electricity generation amount calculation unit 130 calculates the electricity generation amount that is generated by using the methane gas generated by the methane fermentation, based on the generated methane gas amount calculated by the gas amount calculation unit 120 and Expression (3) described below. In the case where the methane gas amount that is increased when the methane fermentation is performed with the addition of the additional material is calculated, the electricity generation amount calculation 10 unit 130 may calculate a difference in electricity generation amount due to the increase in methane gas amount.

$$\begin{matrix}Expression 3 & \\ Electricity Generation Amount kWh = Total Methane ( {m}^{3} ) \times J \times D \times E & ( 3 )\end{matrix}$$

Here, J is an electricity generation amount (kcal/m³) of methane. D is an electricity generation efficiency (%). E is a calorie conversion value (kcal/kWh). Further, the electricity generation amount calculation unit 130 may further calculate an electricity sale income based on the calculated electricity generation amount. The electricity sale income is the amount of income that is expected to be obtained by selling the generated 20 electric power. In the case where the methane gas amount that is increased when the methane fermentation is performed with the addition of the additional material is calculated, the electricity generation amount calculation unit 130 may calculate a difference in electricity sale income due to the increase in methane gas amount.

The compatibility estimation unit 140 estimates a kind of additional material that has high compatibility with the high-nitrogen-concentration organic waste. Specifically, based on the additional-material amount calculated by the additional-material amount calculation unit 110, the compatibility estimation unit 140 selects a kind of additional material that minimizes the additional-material amount that needs to be added, as a kind of additional material that has high compatibility with the high-nitrogen-concentration organic waste.

The collection income calculation unit 150 calculates the amount of income that can be obtained when the additional material is collected. Generally, expense is needed for the disposal of the food waste that is discarded from a business operator such as a restaurant. Therefore, in the case where the food waste is used as the additional material, it is possible to receive payment for collection expense of the food waste, from the business operator that discards the food waste. Generally, the collection expense of the food waste is determined on a weight basis. The collection income calculation unit 150 calculates the collection income based on the additional-material amount that is calculated by the additional-material amount calculation unit 110 and a collection income for the additional material that is determined on a weight basis.

The display unit 160 displays pieces of data calculated or estimated in the organic waste treatment system 100, such that a user can visually recognize the pieces of data. Specifically, the display unit 160 may display the following data.
The additional-material amount calculated by the additional-material amount calculation unit 110.
The generated methane gas amount in the case of the methane fermentation of only the high-nitrogen-concentration organic waste and the generated methane gas amount in the case of the methane fermentation of the mixture containing the additional material, which are generated methane gas amounts calculated by the gas amount calculation unit 120.
The generated methane gas amount that is increased by the methane fermentation with the addition of the additional material, which is a generated methane gas amount calculated by the gas amount calculation unit 120
The electricity generation amount due to the generated methane gas and the electricity generation amount due to the increase in generated methane gas, which are electricity generation amounts calculated by the electricity generation amount calculation unit 130
The electricity sale income due to the generated methane gas and the electricity sale income due to the increase in generated methane gas, which are electricity sale incomes calculated by the electricity generation amount calculation unit 130
The collection income calculated by the collection income calculation unit 150
The compatibility with the high-nitrogen-concentration organic waste for each kind of the additional material

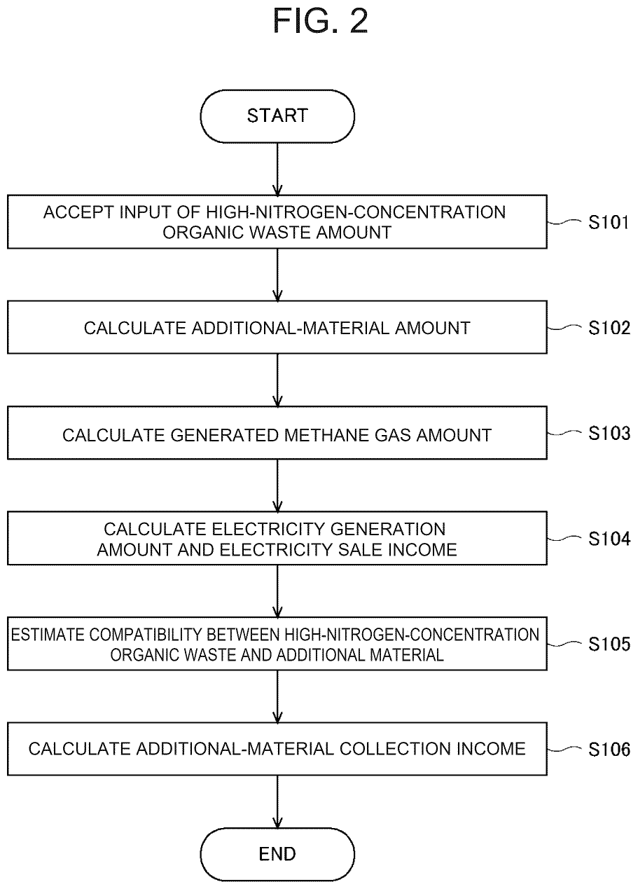

Next, the flow of an organic waste treatment method according to the embodiment will be described with reference to FIG. 2. In the organic waste treatment method according to the embodiment, first, the input of the amount of the high-nitrogen-concentration organic waste to be treated is accepted (step S101). Next, the additional-material amount calculation unit 110 calculates the additional-material amount for each kind of the additional material, based on the high-nitrogen-concentration organic waste amount accepted as the input in step S101 (step S102).

Next, the gas amount calculation unit 120 calculates the methane gas amount that is generated in the case of the methane fermentation of only the high-nitrogen-concentration organic waste accepted in step S101. Further, the gas amount calculation unit 120 calculates the methane gas amount that is generated in the case of the methane fermentation with the addition of the additional material calculated in step S102 (step S103).

Next, the electricity generation amount calculation unit 130 calculates the electricity generation amount and electricity sale income in the case of the electricity generation with the methane gas, based on the generated methane gas amount calculated in step S103. Further, the electricity generation amount calculation unit 130 calculates the electricity generation amount and electricity sale income that are increased in the case of the methane fermentation with the addition of the additional material, based on the generated methane gas amount that is increased in the case of the methane fermentation with the addition of the additional material and that is calculated in step S103 (step S104).

The compatibility estimation unit 140 estimates the compatibility between the high-nitrogen-concentration organic waste accepted as the input in step S101 and each additional material, based on the additional-material amount for each kind of the additional material that is calculated in step S102 (step S105). The collection income calculation unit 150 calculates the collection income in the case where the additional material is collected, based on the additional-material amount calculated in step S102 (step S106). The display unit 160 displays pieces of data calculated or estimated in step S102 to step S106, such that the user can visually recognize the pieces of data (step S107). The user refers to pieces of data displayed on the display unit 160, decides the additional material to be used and the additional-material amount, and performs the treatment of the high-nitrogen-concentration organic waste.

In this way, an appropriate kind and amount of additional material is mixed with the high-nitrogen-concentration organic waste, and the methane fermentation is performed. Thereby, the treatment can be stably performed. Further, since the additional material is the food waste or the chicken eggs, the cost required for the preparation is low. Therefore, the organic waste treatment method according to the embodiment makes it possible to treat the organic waste at low cost.

Next, examples of the present disclosure will be described.

Samples in the organic waste treatment method according to the present disclosure were prepared as follows.

### Raw Materials of Mixture

The organic waste was 1.5 g of the poultry manure. The additional material was the food waste or the chicken eggs. The food waste was the garbage discarded from a restaurant. The organic waste and the additional material were mixed at weight ratios of 1:0, 100:1, 100:5, 10:1, 10:2, and 0:1.

### Fermentation Condition

In the above mixtures, 100 ml of a digestive juice was added, and a mixed fermentation test was performed at 37° C. The digestive juice was a pig manure plant digestive fluid.

### Test Results

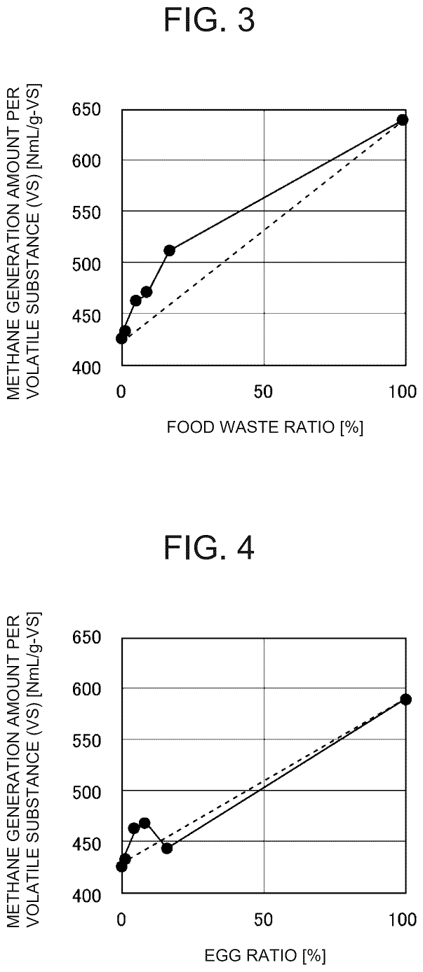

FIG. 3 shows a test result in the case where the additional material was the food waste. FIG. 4 shows a test result in the case where the additional material was the chicken eggs. Each of dotted lines shown in FIG. 3 and FIG. 4 indicates a generated methane gas amount that is theoretically calculated. Each of solid lines shown in FIG. 3 and FIG. 4 indicates a methane gas amount that was actually generated. As shown in FIG. 3, in the case where the food waste was contained in the mixture at a ratio of 5% or more, the methane gas amount that was actually generated was larger than the methane gas amount that is theoretically calculated. Further, as shown in FIG. 4, in the case where the chicken eggs were contained in the mixture at a ratio of less than 20%, the methane gas amount that was actually generated was larger than the methane gas amount that is theoretically calculated. Therefore, it was confirmed that the methane fermentation could be efficiently performed in the case where the food waste was contained in the mixture at a ratio of 5% or more and in the case where the chicken eggs were contained in the mixture at a ratio of less than 20%.

The present disclosure is not limited to the above embodiment, and can be appropriately modified without departing from the spirit.

What is claimed is:

1. An organic waste treatment method of performing methane fermentation of a mixture of poultry manure and food waste, wherein the mixture contains the food waste at a ratio of 5% or more.

2. An organic waste treatment method of performing methane fermentation of a mixture of poultry manure and chicken eggs, wherein the mixture contains the chicken eggs at a ratio of less than 20%.

3. An organic waste treatment system that performs methane fermentation of a mixture of poultry manure and food waste, wherein the mixture contains the food waste at a ratio of 5% or more.
